(19) United States
(12) Patent Application Publication
(10) Pub. No.: US 2007/0065469 A1
(43) Pub. Date: Mar. 22, 2007

(54) Liquid formulations with high concentration of human growth hormone (high) comprising glycine

(76) Inventors: Michael Betz, Schaffhausen (CH); John Stevens, Thornton Heath (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/520,570

(22) PCT Filed: Jul. 8, 2003
(86) PCT No.: PCT/EP03/07346

§ 371 Date: Aug. 16, 2006

(30) Foreign Application Priority Data
Jul. 9, 2002 (US) 60394611
Jul. 9, 2002 (US) 60394612
Jul. 9, 2002 (US) 60394699

(51) Int. Cl. A61K 38/27; A61K 9/00

(52) U.S. Cl. 424/400; 514/12

(57) ABSTRACT

The present invention relates to liquid formulations of human growth hormone (hGH, somatropin) which are storage stable, show reduced or no crystallization on storage and are suitable for administration to the human or animal body. More particularly, the invention relates to liquid formulations of human growth hormone which are stable and exhibit minimal or no crystallization when stored at least for a time at temperatures above refrigeration temperatures.

The present invention relates to liquid formulations of human growth hormone (hGH, somatropin) which are storage stable, show reduced or no crystallization on storage and are suitable for administration to the human or animal body. More particularly, the invention relates to liquid formulations of human growth hormone which are stable and exhibit minimal or no crystallization when stored at least for a time at temperatures above refrigeration temperatures.

Native hGH is a single polypeptide chain protein consisting of 191 amino acids. The protein is internally cross-linked by two disulphide bridges and in monomeric form exhibits a molecular weight of about 22 kDa.

A major biological effect of hGH is to promote growth throughout a range of organs and tissues in the body. hGH is secreted in a pulsatile manner from the pituitary gland throughout life. The major biological effect of hGH is to promote growth. hGH responsive organs or tissues include the liver, intestine, kidneys, muscles, connective tissue and the skeleton. hGH deficiency can occur in all age groups. The consequences of hGH deficiency include reduction in bone density, shortness in stature in children, reduction in lean body mass and extracellular volume and increase in cardiovascular risk factors. Replacement therapy with recombinant hGH has proven safe and effective in reversing these effects, but requires repeated injections at regular intervals

For example, hypopituitary dwarfism is a condition which is readily treated by administering hGH to a subject suffering the condition. Prior to the production of large quantities of hGH by recombinant means only limited amounts of hGH could be prepared by laborious extraction of pituitary glands from human cadavers. This practice carried with it risks associated with infectious agents, eg the agent responsible for Creutzfeldt-Jakob disease (CJD), and that these agents might be passed to the patient receiving hGH. The isolation of the hGH gene and the construction of transformed host cells expressing recombinant hGH in cell culture has opened up not only a more reliable, safer and more cost effective treatment of hypopituitary dwarfism, but the possibility of using hGH for treatment of other diseases and conditions as well. Accordingly, in the context of the present invention, hGH preferably designates recombinant human growth hormone. However, it will readily appreciated that also human growth hormone isolated from natural sources can in principle likewise be included in a pharmaceutical formulation of the present invention.

A long appreciated problem with aqueous liquid formulations of pharmaceutical proteins, not just hGH, is that of instability during storage over a period of time. hGH in aqueous solution is known to undergo a variety of degradative changes. In common with most other proteins, Somatropin (recombinant human growth hormone, rhGH) has three main potential routes of degradation, namely hydrolysis leading to deamidation of free amide groups, oxidation of sulphur containing amino acids, and physical change of aggregation, where two or more hGH molecules physically stick together, for example, resulting in the formation of opaque insolubles. There is also the possibility of a clipping of the peptide backbone as a result of hydrolysis. Additionally, a major problem is crystallization of hGH.

Early suggestions about how to solve the problems of instability noted above included freeze drying, but this of course meant that the resulting lyophilised product needed reconstitution immediately or shortly prior to administration. In the circumstances of routine self-administration by a patient at home, this normally means that the patient has the task of reconstituting the lyophilised preparation into an aqueous solution. This is inconvenient for the patient and carries with it a risk of improper reconstitution due to lack of care, lack of attention to detail and instructions, or simply misunderstanding on the part of the patient. Freeze drying of formulations also suffers from the disadvantage of being costly and time consuming from a manufacturing perspective.

Much effort is therefore expended in finding formulations which permit a simpler self-administration of hGH by patients. These efforts are focused on ways of providing sufficiently stable aqueous liquid hGH formulations in a ready to use form. Such liquid dosage forms offer increased convenience and hence better compliance compared to lyophilized dosage forms which have to be reconstituted and filled into a pen cartridge via an additional device.

However, care has to be taken that excipients which may be able to stabilize an aqueous formulation of hGH may carry some risk in administration to patients. Many compounds which may serve as stabilizers would not appear clinically acceptable and therefore would not enable a pharmaceutically acceptable formulation to be made. Furthermore, pharmaceutical regulatory requirements dictate that any unnecessary additives/excipients, particularly synthetic additives/excipients, must be avoided in order to reduce risks to patients.

Conveniently, aqueous pharmaceutical formulations of hGH should be offered as multi-dosage formulations to the patient, who will administer such a formulation by means of an injector device. Such multi-dosage pharmaceutical formulations usually require an appropriate preservative to be present.

Common liquid formulations of hGH are known to contain the drug at a low concentration, e.g. about 3.33 mg/ml, which, however, upon administration may cause certain disadvantages for the patient.

In particular, a patient has to receive a relatively large volume of such a low-concentration formulation of hGH per injection, which may cause discomfort or even pain. For example, for children suffering from growth hormone deficiency (GHD) hGH may have to be administered at a dosage of about 0.1 IU/kg bodyweight/day. Accordingly, a patient having a bodyweight of 50 kg would have to receive about 5 IU hGH per day, which is contained in 500 μl of a liquid formulation comprising about 3.33 mg/ml hGH (1 IU hGH=0.33 mg hGH). It will readily be appreciated that the application of a volume of less than 500 μl would be highly desired.

In the alternative, such a dosage could be administered in 2 or more injections of such a low-concentrated hGH formulation, each injection having a reduced volume. However, in terms of application safety, the use of more than one injection per dosage is not recommended.

Furthermore, depending on the treatment schedule and dosage, a patient may have to use more than one single injection of such a low-concentration hGH formulation in order to be able to provide the prescribed amount of hGH. This may apply for example to patients having growth deficiency related to the Turner-Syndrome, who because of their increased body weight may be in need of a high amount of hGH. In many instances it will not be possible to deliver the required amount of hGH to such patients with a single injection having a reasonable volume of a such low-concentrated hGH formulation.

Therefore, there is an ongoing need for a liquid pharmaceutical formulation containing hGH at a high concentration.

In the course of the present invention it has been noticed that crystals tend to form in known aqueous, liquid growth hormone formulations if the concentration of hGH is adjusted to higher values, e.g. to 5 mg/ml hGH or more, in such formulations. This does not only apply just when such formulations are stored at refrigeration temperatures, but also when they are stored above refrigeration temperatures, at least for a time. The presence of crystals in liquid hGH formulations is highly undesirable because prior to administration such formulations need to be agitated or swirled and there may be instances when crystals are small or unobserved and the formulation is caused to be administered without dissolving the crystals sufficiently first. There is also the obvious disadvantage in terms of the visual appearance of hGH formulations when crystals have formed during storage.

An object of the invention is therefore to provide a multi-dosage, aqueous liquid hGH formulation which is stable when stored for periods of time at refrigeration temperatures, e.g. for several months, or even for 1 or 2 years. Another object of the invention is to provide liquid hGH formulations which are stable when stored for at least a period of time above common refrigeration temperatures (e.g. above 2° C.-8° C.) or even outside a refrigerator, e.g. for periods of several hours, days, or even weeks.

In the context of the present application, “stable” mainly means that the problem of crystal formation is essentially avoided; preferably this problem is avoided completely. Accordingly, pharmaceutical formulation of the present invention exhibit minimal or no crystallization upon storage as described above.

In addition to avoiding crystallization, a stable formulation should preferably show no or minimal aggregation of hGH upon storage. Likewise, a stable formulation preferably should not or only to a minimal extent undergo other degradation of hGH, e.g. by deamidation, oxidation and/or hydrolysis.

In the context of the present invention, it has been developed that the presence of glycine to be used in such a multi-dosage liquid formulation containing a high concentration of hGH is a favourable parameter regarding stability. Furthermore, in the context of the present invention, it has been surprisingly established that a stable formulation can be composed of a smaller number of excipients than previously thought.

Accordingly, an embodiment of the present invention relates to the use of glycine in the preparation of a multi-dosage aqueous liquid pharmaceutical formulation comprising a high concentration of human growth hormone, as described herein. Preferably, in the pharmaceutical formulations of the present invention, glycine may mainly act as as a stabilizing agent and/or as tonicity-adjusting agent for bringing about a desired tonicity.

In the context of the present invention, a liquid pharmaceutical formulation is a formulation provided in a ready-to-use form, i.e. it is not provided in a form to be reconstituted before administration, like e.g. a lyophilisate.

The present invention therefore provides a multi-dosage liquid pharmaceutical formulation of human growth hormone consisting essentially of human growth hormone at a concentration of from about 5 mg/ml to about 100 mg/ml, glycine, an aqueous buffer, a non-ionic surfactant and a preservative, said pharmaceutical formulation having a tonicity of from about 100 to about 500 mosm/kg and having a pH of from about 6.1 to about 6.3.

Notwithstanding the fact that the further excipients of the pharmaceutical formulation contribute by themselves to the overall tonicity of the formulation, in particular glycine is present such that the tonicity is from about 100 to about 500 mosm/kg. Preferably, the pharmaceutical formulation of the present invention is isotonic, and the amount of glycine present in the formulation will be chosen accordingly.

During the development of the present invention it has been shown that glycine is capable of providing the desired tonicity and simultaneously the desired stability of the pharmaceutical formulation without the need of an additional tonicity-adjusting agent to be present, thereby keeping the overall number of excipients to be used to a minimum.

In the context of the present invention, the term “consisting essentially of” means that the pharmaceutical formulation of the present invention does not contain further excipients, besides the ones mentioned herein, which are capable to contribute a technological pharmaceutical function to the pharmaceutical formulation, e.g. in terms of stability, pH, tonicity, and the like. This does, however, not exclude the possibility that such a formulation may comprise one or more further auxiliary agents, which do not perform a technological pharmaceutical function in the formulation. Such auxiliary agents for example may be pharmaceutically acceptable dyes which will make the liquid formulation coloured. This may e.g. help in identifying the amount of liquid in a multi-dosage injection device or assist in easily identifying whether or not crystallization has occurred.

Arising out of the present invention the inventors have perceived an advantage for patients, pharmacists and medical practitioners. Hitherto it has been necessary to ensure careful storage of growth hormone formulations at refrigeration temperatures (e.g. in the range of 2° to 8° C.) in order to minimize crystallization. Prior to receipt of the growth hormone by patients the formulations can usually be reliably stored at refrigeration temperatures by manufactures and pharmacists. However, once received and stored by patients in domestic refrigerators there is much less reliability in terms of storage temperature. Temperatures in patients' domestic refrigerators may well be substantially above 2-8° C., e.g. be about 15° C., e.g. because of frequent opening. Moreover, devices containing the liquid formulation to be applied may stored outside the refrigerator, e.g. being forgotten on the kitchen bench after administration, thereby being exposed to room temperature (e.g. about 20° C. to about 27° C., frequently about 25° C.) for some time. Crystallization of hGH tends to occur more readily at temperatures greater than 8° C., i.e. above refrigeration temperatures, with known pharmaceutical formulations of hGH.

The formulations of the present invention provide a greater resistance to crystallization if stored for a time above refrigeration temperatures. This therefore permits patients to be supplied with sufficient growth hormone to provide daily doses over longer periods of time than was hitherto recommendable or desirable. Whereas before, patients might have kept a small number of doses for use over a period of a week, with the formulations of the present invention patients may keep several weeks or even several months supply of growth hormone in domestic refrigerator with no or only minimal crystallization taking place. The frequency of prescription to patients can therefore be reduced significantly by the present invention.

Accordingly, the pharmaceutical formulations of the present invention are stable, in particular substantially free of crystallization, on storage at temperatures from refrigeration temperatures to room temperature. In particular, such formulations are stable upon storage at temperatures from refrigeration temperatures to room temperature for at least 4 weeks or at least 1 month, preferably for at least 7 weeks, more preferably for at least 13 weeks, even more preferred for at least 19 weeks. In a preferred embodiment thereof, such formulations are stable, in particular substantially free of crystallization, upon storage at temperatures between 2° C.-8° C. for several months, e.g. for 3 months, preferably for at least 12 months, most preferably for at least 18 months. In a further preferred embodiment thereof, such formulations are stable, in particular substantially free of crystallization, at temperatures between 15° C. and 25° C. for at least 19 weeks.

In this context, it is to mention that prior to storage, hGH formulations may comprise about 4% of “related proteins” being proteinaceous materials generated by degradative processes of deamidation and oxidation. Such “related proteins” are defined in the European Pharmacopoeia and measured by reversed phase HPLC. The inventors propose a maximum of 20% “related proteins” as a target at the end of the shelf life of the formulations.

The degradation rate of hGH is not exactly linear and the rate of degradation increases with an increase in temperature. At 2°-8° C. formulations usually exhibit an increase in “related proteins” of about 0.8% per month. At 25° C. this rises to about 13% per month, and at 40° C. to about 70% per month. Storage at 25° C. for 1 month is approximately equivalent to 17 months storage at 20-8° C. Storage at 15° C. for 1 month is approximately equivalent to 5 months storage at 20-8° C. Continuous storage at a temperature in the range of about 250 to 40° C. is therefore impractical.

Although the formulations of the present invention offer good resistance to crystallization even up to 40° C., particularly up to 25° C., more particularly up to 15° C., the rapid formation of “related proteins” at these temperatures will usually place a more immediate limit on the potential shelf life of formulations.

Rates of “related proteins” formation at different temperatures over time are readily measured by one of average skill and with this information the optimisation and maximum storage time/temperature patterns may be calculated without undue burden. In practice, formulations of the present invention can readily be subjected to a daily rise in temperature slightly above about 8° C. due to the opening and closing of a refrigerator door or removal from a refrigerator for periods of an hour or so each day for the purposes administration without significant loss of shelf life. Advantageously, formulations of the present invention would not suffer adversely in terms of degradation or crystallization if left out of the refrigerator at room temperature for a day or so.

Accordingly, the pharmaceutical formulations of the present invention may be kept at refrigeration temperature (e.g. in the range of 2° to 8° C.) all the time in a stable condition. Furthermore, the pharmaceutical compositions show a sufficient stability when at least some of the overall storage time will be at a temperature above refrigeration temperatures, possibly up to about a week outside a refrigerator, possibly up to about a month or even longer outside a refrigerator.

Accordingly, at least a part of the time that the formulation is stored may be at a storage temperature of at least 8° C., optionally a temperature in the range selected from 8° to 40° C., 8° to 25° C. or 8° to 15° C.

In a preferred embodiment of the pharmaceutical formulations according to the present invention, the concentration of hGH in the formulation is from about 6 mg/ml to about 14 mg/ml. In a particularly preferred embodiment thereof, the concentration of hGH in the formulation is about 6.67 mg/ml.

In the development of the present invention it has surprisingly been established that glycine is capable of providing both sufficient stability to the formulations of the present invention which comprise such a high concentration of hGH as well as substantially contributing the desired tonicity. Preferably, the pharmaceutical formulations of the present invention comprise glycine at a concentration of from about 5 mg/ml to about 75 mg/ml, more preferably from about 5 mg/ml to about 15 mg/ml, most preferably of about 15 mg/ml.

The pharmaceutical formulations according to the present invention preferably may have a tonicity from about 100 mosm/kg to about 500 mosm/kg, i.e. the tonicity of such formulations can be from hypotonic up to hypertonic. In a preferred embodiment thereof, the pharmaceutical formulations of the present invention have a tonicity from slightly hypotonic to slightly hypertonic. Preferably and in accordance with common knowledge (see e.g. Pharmaceutical Dosage Forms, Parenteral Medications, Volume 2; edited by: Kenneth E. Avis; Herbert A. Lieberman; Leon Lachman; Marcel Dekker, Inc. New York and Basel, published: Apr. 1, 1993, page 58-60), this corresponds to a tonicity from about 250 mosm/kg to about 350 mosm/kg. In a particularly preferred embodiment thereof, the pharmaceutical formulations of the present invention are substantially isotonic, preferably isotonic. Isotonicity preferably corresponds to a tonicity of from about 270 mosm/kg to about 328 mosm/kg. More preferably isotonicity corresponds to a tonicity of about 286 mosm/kg. Preferably, the desired tonicity is adjusted with glycine, as outlined herein.

The aqueous buffer present in the pharmaceutical formulation of the present invention can be any pharmaceutically acceptable buffer. Preferred are such aqueous buffers which have in a pharmaceutically sufficient buffer capacity in the desired pH range, i.e. from about 6.1 to about 6.3 and further preferences as disclosed herein. In a preferred embodiment thereof, the aqueous buffer is selected from the group consisting of a phosphate buffer, a citrate buffer, an acetate buffer and a formate buffer, preferably a phosphate buffer, more preferably a sodium phosphate buffer. Usually, the aqueous buffer has a concentration of from about 5 mM to about 100 mM. In a preferred embodiment thereof, the aqueous buffer has a concentration of about 10 mM. In a particularly preferred embodiment thereof, the aqueous buffer is a phosphate buffer having a concentration of about 10 mM (the number 10 mM referring to the concentration of the phosphate ions). Most preferably the aqueous buffer is a sodium phosphate buffer having a concentration of about 10 mM. Likewise preferred is a 10 mM phosphate buffer, in particular a 10 mM sodium phosphate buffer.

The non-ionic surfactant present in the pharmaceutical formulation of the present invention can be any non-ionic surfactant which is pharmaceutically acceptable. Preferably, the non-ionic surfactant is selected from the group consisting of poloxamers, such as poloxamer 184 or 188, and polysorbates such as polysorbate 20 or 80, for example, and other ethylene/polypropylene block polymers. Preferably, the non-ionic surfactant is a poloxamer, in particular poloxamer 188. Amounts of the non-ionic surfactant used may be in the range from about 0.001% (w/v) to about 10% (w/v), more preferably from about 0.005% (w/v) to about 5% (w/v), even more preferably from about 0.01% (w/v) to about 1% (w/v). In a preferred embodiment thereof, the non-ionic surfactant is present at a concentration of from about 0.05 mg/ml to about 4 mg/ml, preferably at a concentration of about 2 mg/ml. A preferred embodiment of the present invention relates to a pharmaceutical formulation wherein the non-ionic surfactant is poloxamer 188 present at a concentration from about 0.05 mg/ml to about 4 mg/ml, preferably of about 2 mg/ml.

The preservative present in the pharmaceutical formulation of the present invention can be any pharmaceutically acceptable preservative. Preferably, the preservative is selected from the group consisting of benzyl alcohol, meta-cresol, methyl paraben, propyl paraben, phenol, benzalkonium chloride, benzethonium chloride, chlorobutanol, 2-phenoxyethanol, phenyl mercuric nitrate and thimerosal. The concentration of the preservative will be readily available to those skilled in the art in agreement with requirements of health authorities regarding the safety of multi-dosage formulations. Accordingly, the concentration of the preservative can be, for example, from about 1 mg/ml to about 30 mg/ml, depending on the preservative actually used. More preferably, the preservative is benzyl alcohol. In a preferred embodiment thereof, the pharmaceutical formulation according to the present invention comprises benzyl alcohol as preservative being present at a concentration of from about 7 mg/ml to 12 mg/ml, most preferably at a concentration of about 9 mg/ml.

In a preferred embodiment, the pH-value of the pharmaceutical formulation according to the present invention is about 6.2. A skilled person would understand a pH of about 6.2 to be from pH 6.15 to pH 6.25. Preferably, the pH is 6.2.

A particularly preferred pharmaceutical formulation of the invention essentially consists of

6.67 mg/ml human growth hormone,

15 mg/ml glycine,

10 mM sodium phosphate buffer,

2 mg/ml poloxamer 188,

9 mg/ml benzyl alcohol,

and has a pH of 6.2.

The crystallization which is minimized or avoided in formulations by the present invention appears to be that of growth hormone. Preferably any crystallization in the liquid formulation is detected directly by eye, more preferably under the light microscope at 5× magnification, even more preferably under the light microscope at lox magnification. Prior to observation under the light microscope formulations may be filtered and the presence or absence of crystals on the filter determined. When viewing under the light microscope the filter may have a pore size of about 5 μm.

A particularly preferred test for crystallization is to store the formulation in a sealed container with no airspace for a time period at 15° C. or at 25° C. in the absence of light and then observe the presence or absence of crystals by eye.

Furthermore, the aqueous growth hormone formulations of the present invention are preferably storage stable in the sense that there is no or minimal aggregation of growth hormone during the period of storage. Also, there is preferably no or minimal chemical degradation of growth hormone, e.g. by deamidation and the like, as described herein. Suitable tests for measuring stability of growth hormone in aqueous solution are well known in the art e.g. as described in WO 94/03198, incorporated herein by way of reference.

In preferred formulations of the present invention, the growth hormone exhibits less than 10% aggregation, preferably less than 1%, more preferably less than 0.1%, even more preferably less than 0.01% aggregation.

In the pharmaceutical formulations according to the present invention, the human growth hormone preferably is recombinantly produced hGH. Accordingly, particularly preferred human growth hormone is produced by recombinant means, for example as taught in EP-A-0 217 822, incorporated herein by way reference. Variants of human growth hormone which may be used in accordance with the invention, alone or in combination with one another and the native hormone, include the 191 amino acid species known as somatropin and the 192 amino acid N-terminal methionine (met) species known as somatrem. There is also the variant known as hGH-V found naturally in the placenta during pregnancy and for which the gene sequence is known and a recombinant protein has been prepared.

The multi-dosage pharmaceutical formulation of the present invention preferably comprises at least two, more preferably a multiplicity of doses of growth hormone.

The amount of hGH in the liquid formulation of the invention depends on the volume of the formulation and the number of doses of hGH that volume is intended to provide. A preferred dosage volume is less than 0.5 ml, like e.g. 0.4 ml, but volumes in the range 0.01 ml to 1.0 ml per single administration may be used in principle. Other preferred dosage volumes may fall in the range 0.1 ml to 0.6 ml, preferably 0.1 ml to 0.4 ml.

In a preferred unit dosage for daily administration the amount of hGH administered is 1.3mg although the precise dosage amount may vary depending on the particular individual. Dosage amounts in the range 0.033 mg to 3.33 mg hGH may be employed, preferably dosages in the range 0.33 mg to 2.0 mg hGH. Increased dosage amounts are appropriate where the frequency of administration is reduced.

The volumes and/or dosage amounts may vary from individual to individual in accordance with specific advice from the clinician in charge.

The pharmaceutical product is preferably in the form of a container for use with an injection device, e.g. a cartridge for use in a pen injector. The pharmaceutical product may be contained within an injection device, preferably a pen injector.

Accordingly, the invention also includes kits comprising an injection device and a separate container containing a liquid growth hormone formulation as hereinbefore described. When the administration device is simply a hypodermic syringe then the kit may comprise the syringe, a needle and a vial or ampoule containing the hGH formulation for use with the syringe. In more preferred embodiments the injection device is other than a simple hypodermic syringe and so the separate container is adapted to engage with the injection device such that in use the liquid formulation in the container is in fluid connection with the outlet of the injection device.

Examples of administration devices include but are not limited to hypodermic syringes and pen injector devices. Particularly preferred injection devices are the pen injectors in which case the container is a cartridge, preferably a disposable cartridge. Accordingly, the invention also provides a cartridge containing any of the liquid formulations as hereinbefore described for use with a pen injector device, the cartridge containing a multiplicity of doses of growth hormone.

The full contents of the texts mentioned are incorporated herein by reference.

The present invention is illustrated in detail by the following examples but is not restricted thereto. In particular, the examples relate to preferred embodiments of the present invention.

### EXAMPLES

The materials mentioned herein, such as reagents, are familiar to the skilled person, commercially available and can be used in accordance with the manufacturer's instructions.

### Example 1

### Preparation and Purification of Bulk Recombinant hGH

Recombinant hGH is produced in cell cultures of CHO cells transformed with the hGH gene to express the hGH protein under culture conditions. Details of how the cells are made and grown are described in EP-A-0 217 822 (Scios Nova), incorporated herein by way of reference. The modification of culture conditions for the growth of cultures on an industrial or commercial scale is well within the abilities of one of average skill in the art.

Once produced by the cells in culture, the hGH needs to be extracted and purified into a form suitable for pharmaceutical use. This is carried out according to the procedures described in AU 629177 (University of New South Wales & Garvan Institute of Medical Research), incorporated herein by way of reference. The resultant hGH preparation is in the form of a bulk solution and this is employed in making the formulations described below. The concentration of hGH in bulk solution (drug substance) usually is from about 8 mg/ml to about 15 mg/ml, for example about 10 mg/ml. Conveniently, the drug substance is present in a 10 mM sodium phosphate buffer.

### Example 2

### Preparation of Human Growth Hormone Formulations

The pharmaceutical formulations are prepared by dilution of a triple concentrated excipient solution to the bulk hGH solution, where necessary adjustment of pH (e.g. with HCl or NaOH), followed by the adjustment of the final weight with water, as outlined in the following.

The bulk hGH solution in 10 mM phosphate can be used either after concentration to values of up to approximately 150 mg hGH/ml or directly at a concentration of, for example, 10 mg hGH/ml. For convenience, the following preparations are performed starting with a bulk hGH solution comprising 10 mg/ml hGH in 10 mM sodium phosphate buffer. If due to different purification steps a bulk hGH solution with a different content of hGH and/or with a different buffer will result, the protocols below will have to be adjusted accordingly. It will be appreciated that such adaption will be well within the routine work of skilled person.

Separately 100 mM solutions of Na₂HPO₄×7 H₂O and NaH₂PO₄×2 H₂O are prepared and mixed with each other to achieve a final pH of 6.2.

6.67 ml of this 100 mM phosphate solution is placed in a beaker for the preparation of 66.67 g triple concentrated excipient solution. The following quantities of excipients are added:

TABLE 1
Composition of triple concentrated excipient solutions

| Composition | | |
|---|---|---|
| Benzyl alcohol | 1.78 | g |
| Poloxamer 188 | 0.40 | g |
| Glycine | 2.96 | g |
| Water for Injection | to 66.67 | g |
| pH | 6.2 | |

The final pharmaceutical formulations is prepared by taking sufficient bulk hGH to give a final concentration of hGH of 6.67 mg/ml. In particular, the preparation comprises placing 32.66 g drug substance (hGH concentration=10 mg hGH/ml) in a beaker. 16.67 g of the triple concentrated excipient solution is added with stirring, where necessary the pH adjusted to a value of 6.2 with HCl or NaOH, and the solution made to 50 g with water.

The solution is filtered via a 0.22 micron filter and filled into cartridges having the plunger stopper already in place. The seal is crimped in place.

The following table shows the final pharmaceutical formulation comprising glycine:

TABLE 2
Composition of the final pharmaceutical formulation:

| | Formulation | |
|---|---|---|
| | 1 | |
| Human Growth Hormone | 6.67 | mg/ml |
| Na₂HPO₄ × 7 H₂O | 0.89 | mg/ml |
| NaH₂PO₄ × 2 H₂O | 1.05 | mg/ml |
| Benzyl Alcohol | 9.0 | mg/ml |
| Poloxamer 188 | 2.00 | mg/ml |
| Glycine | 15 | mg/ml |
| PH | 6.2 | |

Including the phosphate from the hGH bulk solution.

3. Storage of Formulations and Assessment of Crystallization

Cartridges of formulation 1 are stored at 2-8° C., at 15° C. and at 25° C., respectively. The cartridges are examined by eye for the presence or absence of crystals at frequent intervals.

The formulations stored at 2°-8° C. do not show crystallization during the test period. The formulations stored at 15° C. or at 25° C. do not show crystallization for at least 19 weeks.

1. A multi-dosage liquid pharmaceutical formulation of human growth hormone consisting essentially of human growth hormone at a concentration of from about 5 mg/ml to about 100 mg/ml, glycine, a buffer, a non-ionic surfactant, and a preservative, said pharmaceutical formulation having a tonicity of from about 100 to about 500 mosm/kg and having a pH from about 6.1 to about 6.3.

2. The pharmaceutical formulation according to claim 1, wherein the concentration of human growth hormone is from about 6 mg/ml to 14 mg/ml.

3. The pharmaceutical formulation according to claim 2, wherein the concentration of human growth hormone is about 6.67 mg/ml.

4. The pharmaceutical formulation according to claim 1, wherein the concentration of glycine is from about 5 mg/ml to about 75 mg/ml.

5. The pharmaceutical formulation according to claim 1, wherein the concentration of glycine is about 15 mg/ml.

6. The pharmaceutical formulation according to claim 1, said pharmaceutical composition being substantially isotonic.

7. The pharmaceutical formulation according to claim 1, wherein the buffer is selected from the group consisting of a phosphate buffer, a citrate buffer, an acetate buffer and a formate buffer.

8. The pharmaceutical formulation according to claim 7, wherein the buffer is a phosphate buffer.

9. The pharmaceutical formulation according to claim 1, wherein the buffer has a concentration of from about 5 mM to about 100 mM.

10. The pharmaceutical formulation according to claim 1, wherein the buffer has a concentration of about 10 mM.

11. (canceled)

12. The pharmaceutical formulation according to claim 1, wherein the non-ionic surfactant is selected from the group consisting of a poloxamer and a polysorbate.

13. (canceled)

14. The pharmaceutical formulation according to claim 1, wherein the non-ionic surfactant is poloxamer 188.

15. The pharmaceutical formulation according to claim 1, wherein the non-ionic surfactant is present at a concentration of from about 0.05 to about 4 mg/ml.

16. (canceled)

17. (canceled)

18. The pharmaceutical formulation according to claim 1, wherein the preservative is selected from the group consisting of benzyl alcohol, meta-cresol, methyl paraben, propyl paraben, phenol, benzalkonium chloride, benzethonium chloride, chlorobutanol, 2-phenoxyethanol, phenyl mercuric nitrate and thimerosal.

19. The pharmaceutical formulation according to claim 1, wherein the preservative is benzyl alcohol.

20. The pharmaceutical formulation according to claim 1, wherein the preservative is benzyl alcohol being present at a concentration of from about 7 mg/ml to about 12 mg/ml.

21. The pharmaceutical formulation according to claim 1, said pharmaceutical composition having a pH of about 6.2.

22. The pharmaceutical composition according to claim 1, essentially consisting of 6.67 mg/ml human growth hormone,
15 mg/ml propylene glycine,
10 mM sodium phosphate buffer,
2 mg/ml poloxamer 188,
9 mg/ml benzyl alcohol,
and having a pH of 6.2.

23. A kit comprising an injection device and a separate container containing a multi-dosage liquid formulation of human growth hormone according to claim 1.
